(19) United States
(12) Patent Application Publication
(10) Pub. No.: US 2005/0251150 A1
(43) Pub. Date: Nov. 10, 2005

(54) Evulsion aid for fistula catheter and fistula catheter set

(75) Inventor: Taishin Hirano, Kamakura-shi (JP)

Correspondence Address: OSTROLENK FABER GERB & SOFFEN, 1180 AVENUE OF THE AMERICAS, NEW YORK, NY, 100368403, US

(73) Assignee: Olympus Corporation

(21) Appl. No.: 11/011,470

(22) Filed: Dec. 14, 2004

(30) Foreign Application Priority Data
Dec. 15, 2003 (JP) 2003-416525

(51) Int. Cl. A61F 11/00

(52) U.S. Cl. 606/108; 606/167; 604/104

(57) ABSTRACT

This evulsion aid for a fistula catheter has a cylindrical member. The inner diameter of this cylindrical member is larger than the outer diameter of a tube of a fistula catheter, and smaller than the outer diameter of a bumper of the fistula catheter. Moreover, the outer diameter of the cylindrical member is smaller than the fistula. Since the length of the cylindrical member is longer than the fistula, when the cylindrical member is inserted into the fistula, the proximal end of the cylindrical member remains outside the body. When the tube is pulled in the state in which the distal end of the cylindrical member is inserted into the gastric lumen, the bumper is deformed in the direction of contracting diameter by the distal end of the cylindrical member.

### BACKGROUND OF THE INVENTION

1. Field of the Invention

The present invention relates to an evulsion aid used when removing a fistula catheter and an evulsion catheter set that contains the same.

The present application claims priority on Japanese Patent Application No. 2003-416525, filed on Dec. 15, 2003, the content of which is incorporated herein by reference.

2. Description of the Related Art

The ingestion of nutrients using intravenous infusion or the ingestion of nutrients through a tube passed through the nose into the digestive tract are known means for providing nutrients to patients with health disorders and so forth who are unable orally ingest nutrients on their own. However, ingestion of nutrients by these methods frequently subjects the patients to discomfort and pain. Consequently, there have been many cases in recent years in which a method is employed that involves the construction of a fistula (gastrostomy) in the abdomen of the patient using an endoscope, attaching a fistula catheter to this fistula, and ingesting nutrients through this fistula catheter.

Fistula catheters used to ingest nutrients using a fistula are provided with a tube through which nutrients flow, and a bumper attached to the distal end of the tube and implanted in the gastric lumen. The bumper is formed to have a diameter larger than the tube and the fistula to prevent the fistula catheter from being easily removed from the fistula (refer to, for example, US Unexamined Patent Application, First Publication No. U.S. 2003/0109830 A1).

Here, it is necessary to replace this type of fistula catheter at predetermined intervals due to the occurrence of deterioration resulting from continued use. Although there are cases in which an endoscope is used when replacing the fistula catheter, the fistula catheter is frequently extracted transcutaneously.

### SUMMARY OF THE INVENTION

The present invention provides an evulsion aid used for removing a fistula catheter attached to a fistula includes a cylindrical member inserted into the fistula, wherein the inner diameter of the cylindrical member is larger than the outer diameter of a tube of the fistula catheter that passes through the fistula, the outer diameter of the cylindrical member is smaller than the diameter of the fistula, and the length of the cylindrical member is longer than the hole length of the fistula and shorter than the total length of the tube.

In an evulsion aid of the present invention, the distal end of the cylindrical member inserted into the fistula is preferably beveled.

An evulsion aid of the present invention preferably includes a measuring means that measures the amount of insertion of the cylindrical member into the fistula.

In an evulsion aid of the present invention, the measuring means is preferably a scale provided along the longitudinal direction of the cylindrical member.

In an evulsion aid of the present invention, an opening in the distal end of the cylindrical member inserted into the fistula is preferably inclined relative to the plane perpendicular to the longitudinal direction of the cylindrical member.

In an evulsion device of the present invention, at least one cutting edge that cuts the fistula catheter along the direction of evulsion is preferably provided on the inner surface of the cylindrical member.

In an evulsion aid of the present invention, a grasped section is preferably provided on the proximal end side of the cylindrical member, the proximal end being arranged outside the fistula.

In an evulsion aid of the present invention, the cylindrical member has an outer cylinder, and an inner cylinder provided so as to slide freely within the outer cylinder, and a plurality of spreading pieces which deform in the direction of expanding diameter due to their own elasticity when restraint by the outer cylinder is released, are provided on the distal end of the inner cylinder.

The present invention also provides a fistula catheter set including a fistula catheter which includes a tube that passes through a fistula formed between the intestinal wall gastric wall, and a bumper provided on the distal end of the tube that has a section that is larger than the tube; and, a fistula catheter evulsion aid which includes a cylindrical member that has a larger diameter than the tube, a smaller diameter than the fistula, and a length longer than the hole length of the fistula.

### BRIEF DESCRIPTION OF THE DRAWINGS

FIG. 1 is a schematic drawing showing the state in which the fistula catheter has been attached.

FIG. 2 is drawing showing a evulsion aid for fistula catheter in an embodiment of the present invention.

FIG. 3 is a cross-sectional view of the evulsion aid for fistula catheter.

FIG. 4 is a schematic drawing showing the state in which the evulsion aid for fistula catheter has been attached to a fistula.

FIG. 5 is a schematic drawing that explains the process of removing the fistula catheter.

FIG. 6 is a drawing showing a evulsion aid for fistula catheter in an embodiment of the present invention.

FIG. 7 is a cross-sectional view of the evulsion aid for fistula catheter.

FIG. 8 is a cross-sectional view showing the evulsion aid for fistula catheter in an embodiment of the present invention.

FIG. 9 is a cross-sectional view showing a evulsion aid for fistula catheter in an embodiment of the present invention.

FIG. 10 is a drawing that explains the operation of the evulsion aid for fistula catheter.

### DETAILED DESCRIPTION OF THE PREFERRED EMBODIMENTS

The following provides a detailed explanation of preferred embodiments of the invention with reference to the drawings.

First, an explanation is provided of the constitution of a fistula catheter to be removed.

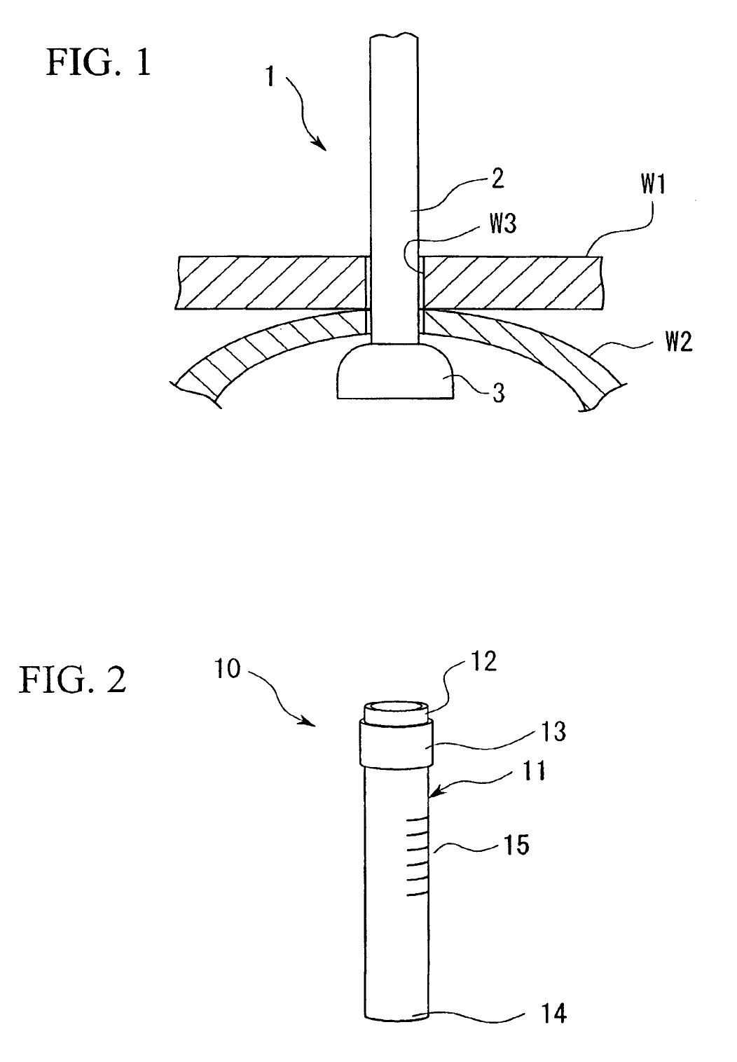

As shown in FIG. 1, a fistula catheter 1 is attached to a fistula (gastrostomy) W3 formed between intestinal wall W1 and gastric wall W2 of a patient. Fistula catheter 1 includes a hollow tube 2 that passes through fistula W3, and a bumper 3 attached to the outer peripheral surface of the distal end of tube 2 and is implanted in the gastric lumen.

Tube 2 has an outer diameter that is smaller than fistula W3 and includes a lumen through which nutrients pass. In addition, this tube 2 can be attached with an adapter (not shown) which has a cap.

Bumper 3 is a hollow member that has the portion attached to tube 2 as its proximal end, and an approximately cup-like shape such that the outer diameter increases moving from the proximal end towards the opening of the distal end. The maximum outer diameter of bumper 3 is larger than fistula W3, and prevents fistula catheter 1 from being removed from fistula W3 during normal use. Bumper 3 is made of silicon resin and so forth, and can be deformed by external force. Furthermore, the shape of bumper 3 shown in FIG. 1 is only one example, and other shapes may be employed.

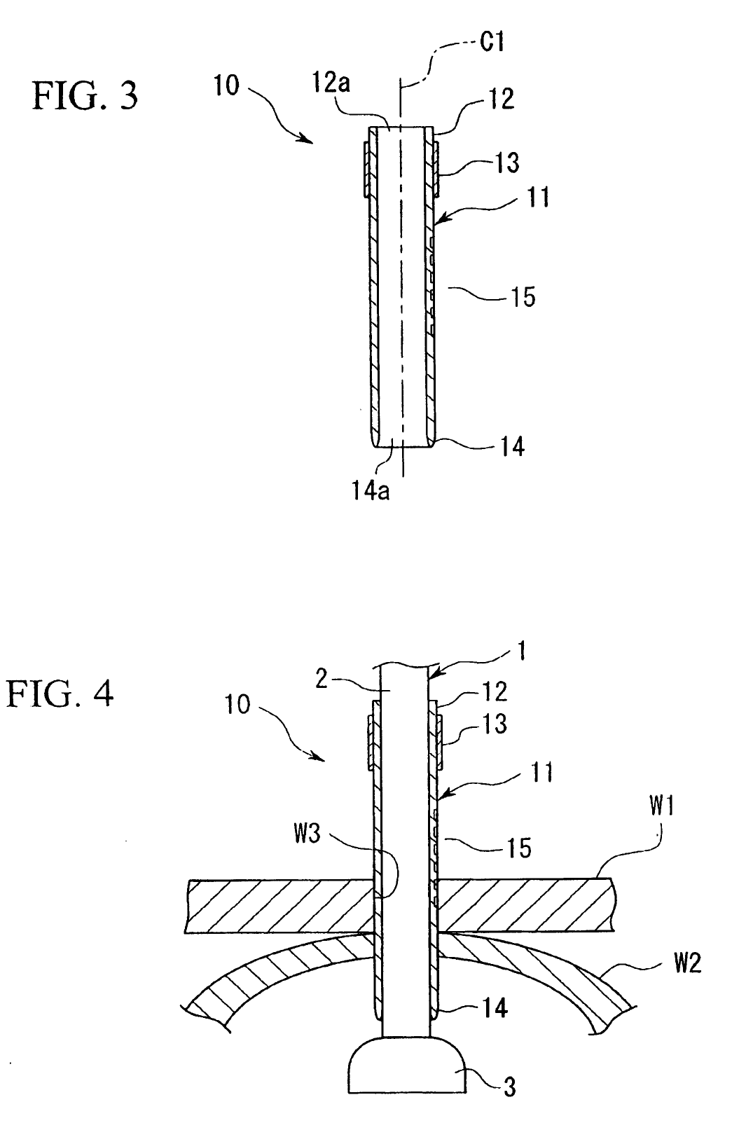

Next, an explanation is provided of the construction of an evulsion aid for a fistula catheter (to simply be referred to as an evulsion aid) in a first embodiment. As shown in FIGS. 2 and 3, evulsion aid 10 includes a cylindrical member 11 and a grasped section 13 provided on the side of proximal end 12 of cylindrical member 11.

Proximal end 12 of cylindrical member 11 positioned at the top of FIGS. 2 and 3 is arranged outside the body. Moreover, distal end 14 positioned at the bottom of those drawings is inserted into the gastric lumen. The length of cylindrical member 11 is longer than the hole length of fistula W3 and shorter than tube 2 of fistula catheter 1. In addition, the inner diameter of cylindrical member 11 is larger than the outer diameter of tube 2 of fistula catheter 1 (see FIG. 1) and smaller than the outer diameter of bumper 3. Moreover, the outer diameter of cylindrical member 11 is approximately equal to the inner diameter of fistula W3 or smaller than the diameter of fistula W3. Furthermore, since the inner diameter of fistula W3 is about 8 mm in the case, for example, the outer diameter of tube 2 of fistula catheter 1 has a diameter of about 7 mm, the wall thickness of cylindrical member 11 is a maximum of about 0.5 mm.

A scale 15 is provided on the outer peripheral surface of cylindrical member 11. Scale 15 is for visually confirming the amount of insertion of evulsion aid 1 when inserted into fistula W3. As shown in FIG. 2, a plurality of lines engraved at predetermined intervals in a portion of the outer peripheral surface of cylindrical member 11 along the longitudinal direction of cylindrical member 11 are shown as an example of scale 15.

In addition, as shown in FIG. 3, the length of distal end 14 of cylindrical member 11 which is inserted into the gastric lumen, in the longitudinal direction of cylindrical member 11 (direction parallel to axis C1) is approximately equal across the circumferential direction of cylindrical member 11. Moreover, distal end 14 is beveled. Consequently, distal end 14 is rounded, and the cross-section along the radius of cylindrical member 11 has a curved shape. Furthermore, cylindrical member 11 is made of, for example, silicon resin or a metal such as stainless steel. The material and wall thickness of this cylindrical member 11 are set so that it has higher rigidity than bumper 3.

Grasped section 13 is attached to the side of proximal end 12 of cylindrical member 11. This grasped section 13 is made from a material that is easily grasped by the hands of a nurse or caregiver who is to remove fistula catheter 1. Furthermore, grasped section 13 may be in the form of tape wrapped around cylindrical member 11. In addition, grasped section 13 may also be integrally formed with cylindrical member 11.

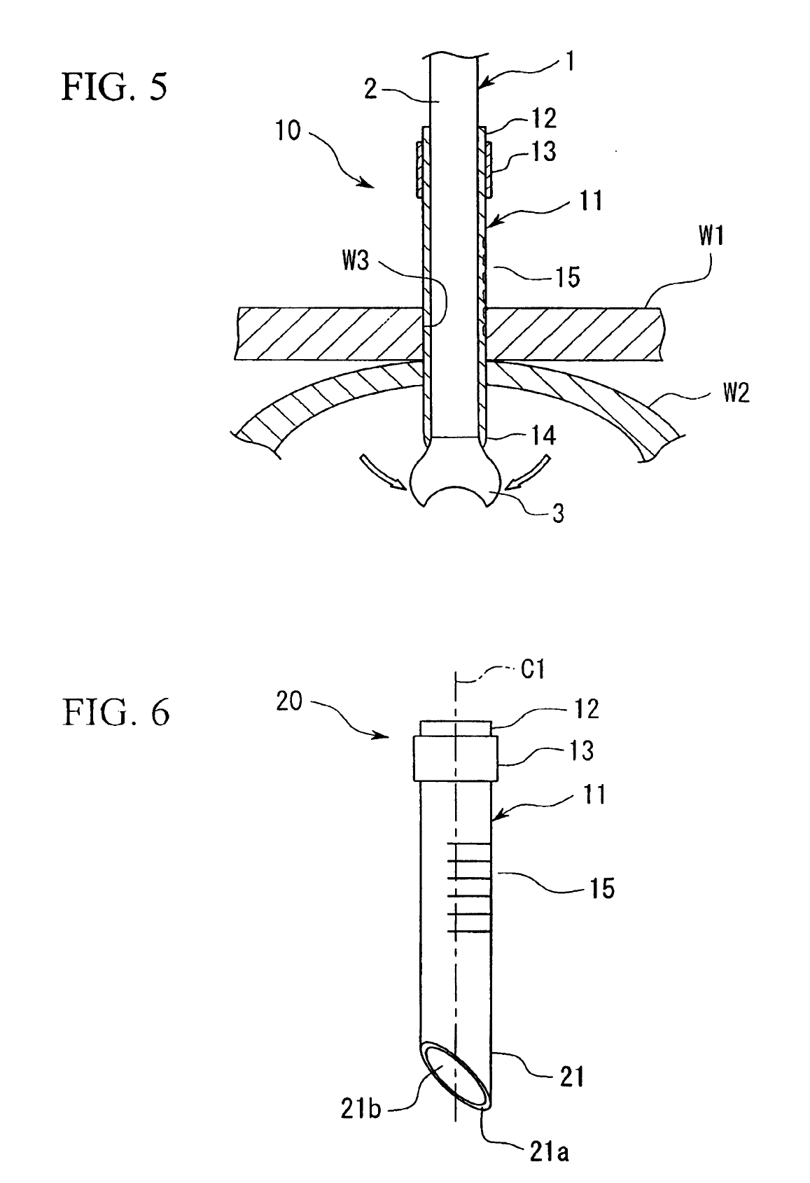

Next, an explanation is provided with reference to FIGS. 4 and 5 of the procedure for removing fistula catheter 1 attached to fistula W3 of a patient as shown in FIG. 1 with evulsion aid 10.

First, tube 2 of fistula catheter 1 is pushed in towards the gastric lumen to form a gap between gastric wall W2 and bumper 3.

Subsequently, tube 2 of fistula catheter 1 is inserted through opening 14a (see FIG. 2) of distal end 14 of cylindrical member 11 while grasping grasped section 13 of evulsion aid 10 and pulled out through opening 12a (see FIG. 2) of proximal end 12. Evulsion aid 10 is then fed towards the gastric lumen along tube 2, and as shown in FIG. 4, distal end 14 of evulsion aid 10 is pushed in between tube 2 and fistula W3. The amount that evulsion aid 10 is inserted at this time can be confirmed with scale 15 on the outer peripheral surface of cylindrical member 11.

Once distal end 14 of evulsion aid 10 has been inserted into the gastric lumen, tube 2 of fistula catheter 1 is pulled along its longitudinal direction without changing the position of evulsion aid 10. Whereupon, as shown in FIG. 5, bumper 3 is pulled in the direction of outside the body together with tube 2. At this time, bumper 3 is deformed in the direction of contracting diameter as indicated with the arrows in FIG. 5 by distal end 14 of cylindrical member 11. As a result of the diameter of bumper 3 being contracted, it is pulled inside cylindrical member 11 and pulled outside the body through cylindrical member 11.

According to this embodiment, since the diameter of bumper 3 of fistula catheter 1 is contracted in the gastric lumen as a result of inserting cylindrical member 11 into fistula W3, fistula catheter 1 is easier to remove. In particular, in contrast to conventional evulsion involving contracting the diameter of bumper 3 by causing it to contact gastric wall W2, since bumper 3 is deformed by causing it to contact distal end 14 of cylindrical member 11, bumper 3 can be easily removed without applying a load to gastric wall W2 surrounding fistula W3.

In addition, since distal end 14 of cylindrical member 11 is beveled, evulsion aid 10 can be inserted easily when inserting into fistula W3. Here, beveled distal end 14 also functions as a guide that aligns the distal end of bumper 3 with the direction of contracting diameter during evulsion of fistula catheter 1.

Moreover, since scale 15 is provided on the outer peripheral surface of cylindrical member 11, the amount evulsion aid 10 is inserted can be easily adjusted.

Evulsion aid 10 also facilitates the insertion and holding of evulsion aid 10 since it includes grasped section 13. Thus, even caregivers having weak hand strength can also perform evulsion easily.

Furthermore, the construction of evulsion aid 10 is only required to include at least one of scale 15, beveled distal end 14 and grasped section 13. Even in such a case, since the use of this evulsion aid enables bumper 3 to contract in diameter without having to push bumper 3 against gastric wall W2, fistula catheter 1 can be removed easily.

In addition, fine indentations may be provided in the inner peripheral surface of cylindrical member 11 so as to be given, for instance, a matte finish or a plurality of grooves may be formed therein along axis C1. When this type of inner peripheral surface is employed, since the contact surface between the inner surface of cylindrical member 11 and fistula catheter 1 can be reduced, fistula catheter 1 can be smoothly passed through cylindrical member 1. Furthermore, an oil, gel or other lubricant may be applied to the inner peripheral surface of cylindrical member 11 instead of or in combination with reducing the contact resistance between evulsion aid 10 and fistula catheter 1 by employing a special shape for the inner peripheral surface.

Moreover, a measuring means used to adjust the amount of insertion of evulsion aid 10 may also be lines imprinted at predetermined intervals along its longitudinal direction. In addition, cylindrical member 11 may also be color-coded corresponding to the distance from distal end 14 or numbers indicating the distance from distal end 14 may be imprinted on cylindrical member 11.

Next, an explanation is provided of a second embodiment of this invention with reference to the drawings. Furthermore, the same reference symbols are used to indicate those constituents of the second embodiment that are the same as those of the first embodiment, and their duplicate explanations are omitted.

As shown in FIG. 6, an evulsion aid 20 of this embodiment has a grasped section 13 and a cylindrical member 11. Distal end 21 of cylindrical member 11 has a predetermined incline angle relative to the plane perpendicular to axis C1 in the lengthwise direction of cylindrical member 11.

During evulsion of fistula catheter 1 (see FIG. 1), tube 2 is inserted through opening 21b of distal end 21 and then pulled out through opening 12a of proximal end 12 (see FIG. 3). Moreover, evulsion aid 20 is inserted from the most protruding section 21a of distal end 21 into fistula W3, and inserted until the entirety of distal end 21 reaches the gastric lumen. The amount of insertion at this time is confirmed with scale 15. When tube 2 of fistula catheter 1 is then pulled, bumper 3 is deformed in the direction of contracting diameter by distal end 21, and fistula catheter 1 is removed from fistula W3.

According to this evulsion aid 20, since distal end 21 of cylindrical member 11 is inclined, it is easily inserted into fistula W3. In addition, since opening 21b is inclined, the surface area of the opening is larger thereby facilitating incorporation of bumper 3 (see FIG. 1). Moreover, since a portion of bumper 3 is pressed by the distal end of opening 21b, stress is focused at a single point making it easier to collapse bumper 3. Thus, fistula catheter 1 can be removed more easily. Furthermore, inclined distal end 21 also preferably has a curved shape as shown in FIG. 3 resulting from beveling.

Next, an explanation is provided of a third embodiment of this invention with reference to the drawings. Furthermore, the same reference symbols are used to indicate those constituents of the third embodiment that are the same as those of each of the aforementioned embodiments, and their duplicate explanations are omitted.

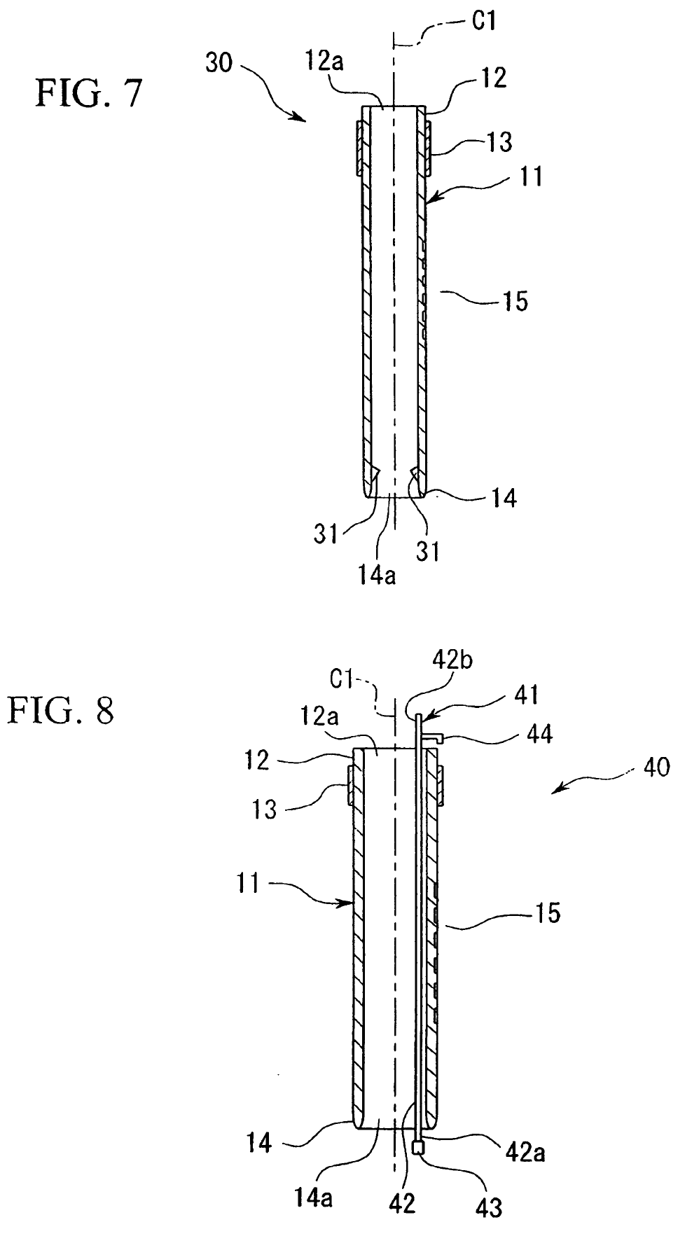

As shown in FIG. 7, an evulsion aid 30 in this embodiment has a grasped section 13 and a cylindrical member 11. Moreover, two cutters 31 are attached to the inner peripheral surface of cylindrical member 11.

Cutters 31 are attached so as to oppose each other at locations having an angle of about 180° from each other around axis C1 in the vicinity of distal end 14 of cylindrical member 11. The cutting edges of cutters 31 are facing downward, namely are facing towards opening 14a of distal end 14. Furthermore, cutters 31 may be attached directly to cylindrical member 11, or they may be provided in rings that engage with the inner peripheral surface of cylindrical member 11. Moreover, cutters 31 may be provided with a metal plate attached to cylindrical member 11 so as to follow along the inner peripheral surface.

In this evulsion aid 30, tube 2 of fistula catheter 1 as shown in FIG. 1 is inserted into fistula W3 so as to pass through it. At this time, cutters 31 arranged on the inside of cylindrical member 11 cut at two locations of tube 2 and separate tube 2 into two parts. When tube 2 is pulled after inserting distal end 14 of evulsion aid 30 into the gastric lumen, together with bumper 3 being deformed in the direction of contracting diameter by distal end 14 of cylindrical member 11, it is pulled outside the body. At this time, bumper 3 that is pulled into cylindrical member 11 is pulled outside the body while being cut approximately along in the direction in which it is pulled by cutters 31.

According to this embodiment, since bumper 3 can be cut approximately along the direction in which it is pulled by cutters 31 during evulsion of fistula catheter 1, it becomes easier to reduce the outer diameter of bumper 3. Thus, fistula catheter 1 can be removed without having to pull on tube 2 with a large force. Here, since cutters 31 are arranged in the longitudinal direction of cylindrical member 11, namely parallel to the direction of evulsion of fistula catheter 1, there is no risk of tube 2 or bumper 3 falling off and remaining in the gastric lumen.

Furthermore, the number of cutters 31 may also be one or three or more. In the case of three or more cutters 31, cutters 31 are preferably arranged at equal intervals along the inner peripheral surface of cylindrical member 11.

Next, an explanation is provided of a fourth embodiment of this invention with reference to the drawings. Furthermore, the same reference symbols are used to indicate those constituents of the fourth embodiment that are the same as those of each of the aforementioned embodiments, and their duplicate explanations are omitted.

As shown in FIG. 8, an evulsion aid of this embodiment includes a cylindrical member 11, a grasped section 13 and a sensor (measuring means) 41 that measures the amount the cylindrical member is inserted into fistula W3.

Sensor 41 has a rod 42 that passes through cylindrical member 11 parallel to its axis C1, a contact section 43 attached to distal end 42a of rod 42, and an engaging section 44 attached to proximal end 42b of rod 42.

The length of rod 42 is longer than cylindrical member 11. Distal end 42a of rod 42 protrudes beyond distal end 14 of cylindrical member 11. Engaging section 44 extends from proximal end 42b of rod 42 in the direction parallel to the radial direction of cylindrical member 11. Moreover, the distal end of engaging section 44 is bent so as to be able to engage with proximal end 12 of cylindrical member 11. In the state prior to inserting evulsion aid 40 into fistula W3, engaging section 44 engages with the upper surface of proximal end 12 of cylindrical member 11, to prevent sensor 41 from falling out of cylindrical member 11.

The following provides an explanation of the operation of this embodiment.

Initially, evulsion aid 40 is inserted into fistula W3 while passing tube 2 through cylindrical member 11. At this time, contact section 43 of sensor 41 is also led to the gastric lumen accompanying insertion of cylindrical member 11 into fistula W3. When contact section 43 of sensor 41 contacts bumper 3 from the direction outside the body, sensor 41 cannot be inserted any further. When cylindrical member 11 is continued to be inserted from this position, since sensor 41 does not move, the side of the proximal end 42b of sensor 41 protrudes from cylindrical member 11.

In other words, if the amount of insertion of evulsion aid 40 when rod 42 protrudes from cylindrical member 11 is preset to the length of rod 42 so that the amount of insertion is suitable for evulsion, the amount of insertion of evulsion aid 40 can be adjusted to a suitable value by the movement of sensor 41. Furthermore, a through hole (not shown) may be provided in parallel with axis C1 in cylindrical member 11, and rod 42 may be allowed to pass through that through hole while sliding freely.

Next, an explanation is provided of a fifth embodiment of this invention with reference to the drawings. Furthermore, the same reference symbols are used to indicate those constituents of the fifth embodiment that are the same as those of each of the aforementioned embodiments, and their duplicate explanations are omitted.

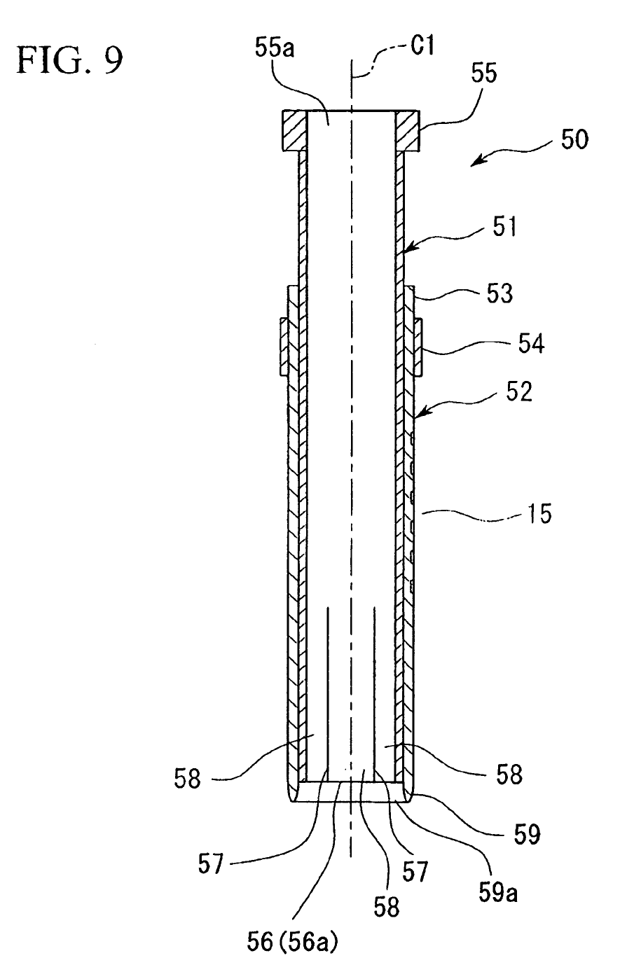

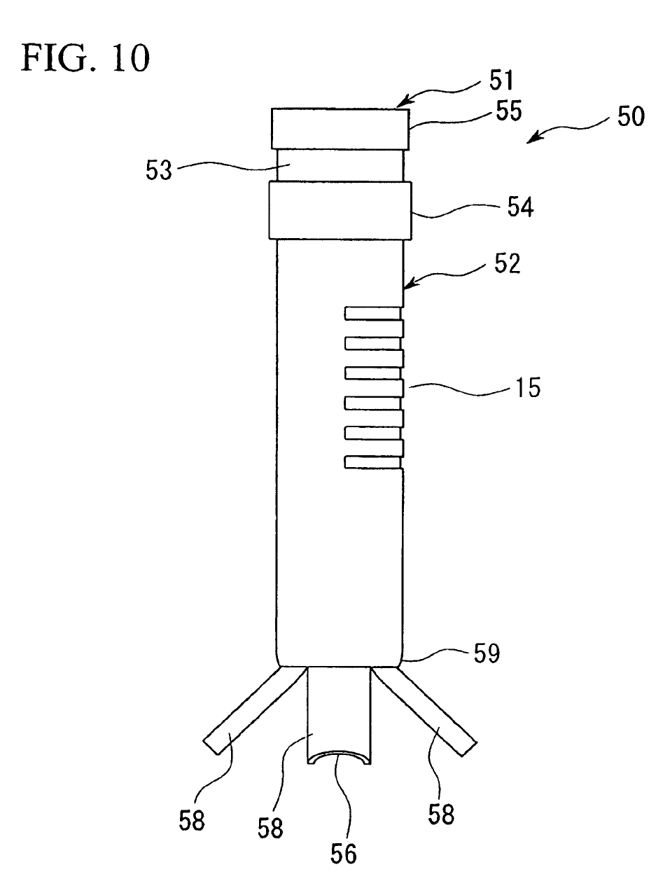

As shown in FIG. 9, an evulsion aid 50 in this embodiment includes an inner tube 51 having an inner diameter approximately equal to the outer diameter of tube 2 (see FIG. 1) of fistula catheter 1, an outer tube 52 that makes sliding contact with the outer peripheral surface of inner tube 51, and a grasped section 54 attached to the side of proximal end 53 of outer tube 52.

An inner tube grasped section 55 is formed on the proximal end of inner tube 51.

This inner tube grasped section 55 is used for being grasped by a nurse and so forth, and its outer diameter is larger than the outer peripheral surface of outer tube 52. In addition, a plurality of slits 57 are provided parallel to axis C1 and at equal intervals along the circumferential direction of inner tube 51 in the peripheral wall of distal end 56 of inner tube 51. Distal end 56 of inner tube 51 is divided into a plurality of spreading pieces 58 by these slits 57. These spreading pieces 58 are produced to be elastically deformable so that their distal ends open in the state in which inner tube 51 is removed from outer tube 52 (natural state). Furthermore, as shown in FIG. 9, spreading pieces 58 are in the closed state housed within outer tube 52. As an example of a process for producing this type of inner tube 51, a thermoplastic resin may be heat-treated in the state in which the distal ends are spread open or molded by using a mold having a shape in which the distal ends are spread open. Moreover, inner tube 51 may also be produced from a metal material such as stainless steel.

The inner diameter of outer tube 52 is approximately equal to the outer diameter of inner tube 51, and houses inner tube 51 while allowing to slide freely.

Moreover, the outer diameter of outer tube 52 is approximately equal to or smaller than the diameter of fistula W3. A scale 15 composed of a plurality of projections is provided on the outer peripheral surface of outer tube 52. Furthermore, distal end 59 of outer tube 52 is beveled.

When removing fistula catheter 1 using this evulsion aid 50, after inserting tube 2 through opening 59a of distal end 59 of outer tube 52 shown in FIG. 9 and passing it through opening 56a of distal end 56 of inner tube 51, it is pulled out through opening 55a of inner tube grasped section 55 of inner tube 51. Moreover, inner tube 51 and outer tube 52 are then fed simultaneously relative to tube 2 while in this state and inserted in fistula W3. Once evulsion aid 50 has been inserted to a predetermined position by confirming the amount of insertion with scale 15, inner tube 51 is pushed into the gastric lumen while keeping the position of outer tube 52 fixed with grasped section 54.

When distal end 56 of inner tube 51 protrudes beyond distal end 59 of outer tube 52, each spreading piece 58 opens so as to spread towards the direction parallel to the radial direction of inner tube 51 between gastric wall W2 and bumper 3. When tube 2 is pulled while in this state, the opened spreading pieces 58 make contact with bumper 3 and are deformed in the direction of contracting diameter by bumper 3 along the incline of spreading pieces 58. The contracted diameter bumper 3 is then removed outside the body through inner tube 51. Furthermore, once fistula catheter 1 has been removed, inner tube 51 is pulled back causing spreading pieces 58 to be housed in outer tube 52. Evulsion aid 50 is subsequently extracted from fistula W3.

According to this embodiment, since this evulsion aid has an inner tube 51 that spreads into the gastric lumen, bumper 3 can be contracted without contacting gastric wall W2. Thus, fistula catheter 1 can be removed easily. In addition, since evulsion aid 50 has a double-walled structure, spreading pieces 58 can be opened and closed using a simple constitution.

Here, the evulsion may also be composed of a single-walled cylindrical member, and a plurality of spreading pieces provided on the distal end of the cylindrical member may be opened and closed with wires and so forth. In this case, by pulling on the wires by hand or operating a button that controls a device that winds up the wires, since the end pieces can be opened and closed, accordingly it possible to easily remove fistula catheter 1.

Next, an explanation if provided of a sixth embodiment of this invention with reference to the drawings. Furthermore, the same reference symbols are used to indicate those constituents of the sixth embodiment that are the same as those of each of the aforementioned embodiments, and their duplicate explanations are omitted.

This embodiment relates to a fistula catheter set used when constructing a gastrostomy or when replacing a fistula catheter.

This fistula catheter set includes a fistula catheter 1 as shown in FIG. 1, and at least one of evulsion aids 10, 20, 30, 40 or 50 of any of the aforementioned embodiments. Additional constituents of this fistula catheter set include guide wires, snare, syringe, scalpel and needle.

According to this fistula catheter set, a fistula catheter 1 placed in a patient can be removed using evulsion aid 10, 20, 30, 40 or 50 when fistula catheter 1 is to be replaced or when fistula catheter 1 is no longer necessary. Thus, evulsion of fistula catheter 1 is easy.

Furthermore, this invention can be applied in a wide range of other applications without being limited to each of the aforementioned embodiments.

For example, although each of the aforementioned embodiments have explained a fistula catheter that is attached to a gastrostomy, the catheter may also be attached to a fistula formed between an organ such as the intestines and another organ or between an organ and the body surface.

The invention may also be an evulsion aid or fistula catheter set that comprises an evulsion aid by combining the characteristic features of evulsion aids 10, 20, 30, 40 and 50 in each of the embodiments. Specific examples of this include an evulsion aid provided with cutters 31 as shown in FIG. 7 and an inclined distal end 21 as shown in FIG. 6, as well as an evulsion aid in which cutters 31 are provided in an inner tube 52 having a double-walled structure as shown in FIG. 9.

In addition, the distal ends 14, 21 and 59 of evulsion aids 10, 20, 30, 40 and 50 may have two or more gentle surface irregularities along their circumference such that the length in the direction of axis C1 differs cyclically. As a result, bumper 3 is folded up easily since non-uniform stress is applied thereto.

Moreover, in the case a scale is provided on tube 2 of fistula catheter 1, cylindrical members 11, 51 and 52 of evulsion aids 10, 20, 30, 40 and 50 may be made of a transparent material. As a result, the amount of insertion of evulsion aids 10, 20, 30, 40 and 50 can be measured by reading the scale of tube 2 at the position of proximal ends 12 and 53 or grasped sections 13 and 54 of cylindrical members 11, 51 and 52.

In addition, these evulsion aids 10, 20, 40 and 50 can also be used when attaching fistula catheter 1 to fistula W3. More specifically, evulsion aids 10, 20, 40 and 50 are inserted until distal ends 14, 21 and 59 pass through fistula W3 in the state in which bumper 3 is housed within cylindrical member 11 or inner tube 51. Subsequently, after bumper 3 is pushed into the gastric lumen, evulsion aids 10, 20, 40 and 50 are extracted from fistula W3. As a result, since the evulsion aids can be attached in the state in which the diameter of bumper 3 is contracted beyond the diameter of fistula W3, they can be easily attached to fistula W3. In this case, evulsion aids 10, 20, 40 and 50 function as insertion aids of fistula catheter 1.

According to an evulsion aid of the present invention, the bumper implanted in the body is deformed in the direction of contracting diameter by a cylindrical member when a tube of a fistula catheter is pulled after inserting the evulsion aid into the fistula. Thus, the fistula catheter can be removed without causing the bumper to press against the gastric wall.

According to an evulsion aid of the present invention, since a beveled section of the evulsion aid is inserted into a fistula, the evulsion aid is inserted smoothly.

Since an evulsion aid of the present invention includes a measuring means, the amount of insertion into a fistula is easily adjusted to a value suitable for evulsion of a fistula catheter.

According to an evulsion aid of the present invention, the amount of insertion into a fistula can be adjusted while visually confirming a scale.

According to an evulsion aid of the present invention, since the section that is first inserted into a fistula is inclined, the evulsion aid is inserted easily. Moreover, it also becomes easier to collapse and contract the diameter of the bumper of a fistula catheter.

According to an evulsion aid of the present invention, since cutting edges are provided on its inner surface, a portion of a fistula catheter is cut during evulsion. In particular, a bumper is easily deformed in the direction of contracting diameter since a portion of the bumper having a diameter larger than a fistula is cut by the cutting edges.

Since an evulsion aid of the present invention includes an easily grasped section, the evulsion aid can be reliably held during insertion into a fistula and evulsion of a fistula catheter.

As a result of an evulsion aid of the present invention having a double-walled structure, and sliding an inner tube, in which the distal end is formed so as to open, relative to an outer tube, it can be opened and closed inside the body. Consequently, a bumper can be deformed while guiding in the direction of contracting diameter by opened spreading pieces.

A fistula catheter set of the present invention consists of a fistula catheter to be attached to a fistula of a patient, and an evulsion aid combined in the form of a set. Consequently, since the diameter of a bumper can be contracted without contacting the gastric wall by a cylindrical member, the fistula catheter can be removed easily.

According to this invention, since an evulsion aid has a cylindrical member having a diameter smaller than a fistula and larger than a tube of a fistula catheter, when the fistula catheter is pulled out in the state in which this cylindrical member is inserted into the fistula, a bumper of the fistula catheter can be deformed in the direction of contracting diameter by the distal end of the cylindrical member. Thus, since the bumper is not pushed against the body during evulsion of the fistula catheter, the fistula catheter is removed easily.

While preferred embodiments of the invention have been described and illustrated above, it should be understood that these are exemplary of the invention and are not to be considered as limiting. Additions, omissions, substitutions and other modifications can be made without departing from the spirit or scope of the present invention. Accordingly, the invention is not to be considered as being limited by the foregoing description, and is only limited by the scope of the appended claims.

1. An evulsion aid used for removing a fistula catheter attached to a fistula, comprises a cylindrical member inserted into the fistula,
wherein the inner diameter of the cylindrical member is larger than the outer diameter of a tube of the fistula catheter that passes through the fistula, the outer diameter of the cylindrical member is smaller than the diameter of the fistula, and the length of the cylindrical member is longer than the hole length of the fistula and shorter than the total length of the tube.

2. An evulsion aid for a fistula catheter according to claim 1, wherein the distal end of the cylindrical member inserted into the fistula is beveled.

3. An evulsion aid for a fistula catheter according to claim 1, further comprises a measuring means that measures the amount of insertion of the cylindrical member into the fistula.

4. An evulsion aid for a fistula catheter according to claim 3, wherein the measuring means is a scale provided along the longitudinal direction of the cylindrical member.

5. An evulsion aid for a fistula catheter according to claim 1, wherein an opening in the distal end of the cylindrical member inserted into the fistula is inclined relative to the plane perpendicular to the longitudinal direction of the cylindrical member.

6. An evulsion aid for a fistula catheter according to claim 1 wherein, at least one cutting edge that cuts the fistula catheter along the direction of evulsion is provided on the inner surface of the cylindrical member.

7. An evulsion aid for a fistula catheter according to claim 1, wherein a grasped section is provided on the proximal end side of the cylindrical member, the proximal end being arranged outside the fistula.

8. An evulsion aid for a fistula catheter according to claim 1, wherein the cylindrical member has an outer cylinder and an inner cylinder provided so as to slide freely within the outer cylinder, and a plurality of spreading pieces which deform in the direction of expanding diameter due to their own elasticity when restraint by the outer cylinder is released, are provided on the distal end of the inner cylinder.

9. A fistula catheter set comprising:
a fistula catheter which comprises a tube that passes through a fistula formed between the intestinal wall gastric wall, and a bumper provided on the distal end of the tube that has a section that is larger than the tube; and
a fistula catheter evulsion aid which comprises a cylindrical member that has a larger diameter than the tube, a smaller diameter than the fistula, and a length longer than the hole length of the fistula.
